(19) United States
(12) Patent Application Publication
(10) Pub. No.: US 2005/0232986 A1
(43) Pub. Date: Oct. 20, 2005

(54) Dosage form containing promethazine and another drug

(76) Inventors: David Brown, Colleyville, TX (US); Ralph Brown, Southlake, TX (US); Himanshu Patel, North Richland Hills, TX (US); Viswanathan Srinivasan, The Woodlands, TX (US)

Correspondence Address: GREENBLUM & BERNSTEIN, P.L.C., 1950 ROLAND CLARKE PLACE, RESTON, VA, 20191, US

(21) Appl. No.: 10/736,902

(22) Filed: Dec. 17, 2003

(51) Int. Cl. A61K 31/5415; A61K 31/485; A61K 9/20; A61K 9/24

(52) U.S. Cl. 424/464; 514/282; 514/225.8

(57) ABSTRACT

A pharmaceutical dosage form which comprises promethazine and/or a pharmaceutically acceptable salt thereof and at least one second drug. The dosage form provides a plasma concentration within the therapeutic range of the at least one second drug over a period which is coextensive with a substantial part of the period over which the dosage form provides a plasma concentration within the therapeutic range of promethazine or salt thereof. This abstract is neither intended to define the invention disclosed in this specification nor intended to limit the scope of the invention in any way.

### BACKGROUND OF THE INVENTION

1. Field of the Invention

The present invention relates to a pharmaceutical dosage form which contains promethazine and/or a pharmaceutically acceptable salt thereof in combination with at least one additional active ingredient. The dosage form releases promethazine and the additional active ingredient at rates which provide pharmaceutically suitable plasma concentrations of both components over similar periods of time. The present invention also relates to a process for manufacturing the dosage form and to methods for alleviating conditions which can be alleviated by promethazine and the at least one additional active ingredient.

2. Discussion of Background Information

Promethazine hydrochloride is a phenothiazine derivative which possesses antihistaminic, sedative, antimotion-sickness, antiemetic, and anticholineric effects. It is used, for example, for the amelioration of allergic reactions, the treatment of motion sickness and the prevention and control of nausea and vomiting associated with certain types of anesthesia and surgery. Allergic reactions, in particular, which can be treated or ameliorated with promethazine hydrochloride are often accompanied by conditions which can not satisfactorily be ameliorated or treated with promethazine, but may be treated or ameliorated by other drugs, e.g., expectorants, mucus thinning drugs, decongestants, antitussives, analgesics and/or antihistamines. However, a single dose of promethazine hydrochloride can provide a therapeutically effective plasma concentration for an extended period of time, up to 12 hours and even longer, whereas a single does of other drugs will often provide a therapeutically effective plasma concentration for a considerably shorter period. For example, a single dose of an expectorant such as guaifenesin will usually provide relief for only about one hour, and decongestants, antitussives, and analgesics usually provide relief for about 4 to 8 hours per single dose. As a result, there appears to be virtually no benefit in combining promethazine hydrochloride and any such drug with a noticeably shorter effective period in a single dosage form. With a corresponding combination, the promethazine hydrochloride would still provide the desired therapeutic effect when the other drug has long ceased to be effective and would have to be administered again.

It would be desirable if patients suffering from, e.g., respiratory congestion, inflammation of the respiratory mucosa and sinus cavities, weeping eyes, rhinorrhea, Eustachian Tube congestion, cough, nausea, aching joints, headache and fever and related symptoms, for which promethazine is indicated, would also obtain relief, over a comparable time period, from one or more conditions for which drugs different from promethazine are indicated, by administering a single dose of a dosage form such as, e.g., a tablet, liquid, syrup, suspension, capsule and the like which provides both promethazine and one or more other drugs.

### SUMMARY OF THE INVENTION

The present invention provides a pharmaceutical dosage form which comprises a first drug which is selected from promethazine and pharmaceutically acceptable salts thereof, and at least one second drug. The dosage form provides a plasma concentration within the therapeutic range of the at least one second drug over a period which is coextensive with at least about 70% of the period over which the dosage form provides a plasma concentration within the therapeutic range of the first drug.

In one aspect of the dosage form, the at least one second drug is preferably selected from decongestants, antitussives, expectorants, mucus thinning drugs, analgesics and antihistamines. For example, the at least one second drug may comprise one or more antitussives such as, e.g., codeine, dihydrocodeine, hydrocodone, dextromethorphan and pharmaceutically acceptable salts thereof, and/or the at least one second drug may comprise one or more decongestants such as, e.g., phenylepherine, pseudoephedrine and pharmaceutically acceptable salts thereof, and/or the at least one second drug may comprise one or more antihistamines, e.g., chlorpheniramine or a pharmaceutically acceptable salt thereof, and/or the at least one second drug may comprise one or more expectorants, e.g., guaifenesin.

In another aspect of the dosage form of the present invention, the first drug may comprise promethazine hydrochloride.

In yet another aspect, the plasma half-life of the at least one second drug may be shorter than the plasma half-life of the first drug by at least about 3 hours, e.g., by at least about 4 hours, or by at least about 6 hours.

In a still further aspect, the period of a plasma concentration within the therapeutic range of the at least one second drug may be coextensive with at least about 80%, e.g., at least about 90%, or at least about 95%, of the period of a plasma concentration within the therapeutic range of the first drug.

In another aspect, the dosage form may be a tablet. For example, the tablet may have at least two layers. Preferably, the tablet is a bi-layered tablet.

In yet another aspect, the dosage form comprises a solution or a suspension.

The present invention also provides a bi-layered tablet which comprises two layers. The first layer comprises promethazine and/or a pharmaceutically acceptable salt thereof. The second layer comprises at least one additional drug which is selected from decongestants, antitussives, expectorants, mucus thinning drugs, analgesics and antihistamines. The bi-layered tablet provides a plasma concentration within the therapeutic range of the at least one additional drug over a period which is coextensive with at least about 70% of the period over which the bi-layered tablet provides a plasma concentration within the therapeutic range of the promethazine or pharmaceutically acceptable salt thereof.

In one aspect of the bi-layered tablet, the second layer may comprise one or more of phenylepherine, pseudoephedrine, chlorpeniramine and pharmaceutically acceptable salts thereof.

In another aspect, the first layer may comprise promethazine hydrochloride and the second layer may comprise two or more of phenylepherine, pseudoephedrine, chlorpeniramine and pharmaceutically acceptable salts thereof.

In yet another aspect, the first layer comprises promethazine or a pharmaceutically acceptable salt thereof as the only active ingredient. For example, promethazine hydrochloride may be the only active ingredient in the first layer.

In a still further aspect of the bi-layered tablet of the present invention, the period of a plasma concentration within the therapeutic range of the at least one second drug may be coextensive with at least about 80%, e.g., at least about 90%, of the period of a plasma concentration within the therapeutic range of the promethazine or pharmaceutically acceptable salt thereof.

In a still further aspect of the bi-layered tablet, the first layer preferably is an immediate release layer and/or the second layer is a controlled release layer.

In another aspect, the first layer of the bi-layered tablet may contain from about 0.1 mg to about 90 mg, e.g., from about 5 mg to about 60 mg, preferably from about 25 mg to about 50 mg, of promethazine hydrochloride.

In yet another aspect of the bi-layered tablet, the second layer thereof may be a controlled release layer and may contain (i) from about 0.1 mg to about 16 mg of chlorpheniramine maleate or an equivalent amount of any other pharmaceutically acceptable salt of chlorpheniramine; and/or (ii) from about 1 mg to about 90 mg of phenylepherine hydrochloride or an equivalent amount of any other pharmaceutically acceptable salt of phenylepherine; and/or (iii) from about 1 mg to about 240 mg of pseudoephedrine hydrochloride or an equivalent amount of any other pharmaceutically acceptable salt of pseudoephedrine.

The present invention also provides a multi-layered tablet which comprises at least a first layer and a second layer. The first layer comprises promethazine and/or a pharmaceutically acceptable salt thereof and the second layer is a controlled release layer and comprises at least one drug which is selected from decongestants, antitussives, expectorants, mucus thinning drugs, analgesics and antihistamines.

In one aspect, the first layer of the multi-layered tablet may be an immediate release layer. In another aspect, the first layer may comprise promethazine hydrochloride.

In yet another aspect, the first layer may contain promethazine or a pharmaceutically acceptable salt thereof as the only active ingredient.

In a still further aspect of the multi-layered tablet of the present invention, the second layer preferably comprises one or more, e.g., at least two, of codeine, dihydrocodeine, hydrocodone, dextromethorphan, phenylepherine, pseudoephedrine, guaifenesin, chlorpheniramine and pharmaceutically acceptable salts thereof.

In another aspect of the multi-layered tablet, the at least one drug in the second layer may have a plasma half-life which is shorter by at least about 3 hours than the plasma half-life of promethazine and/or pharmaceutically acceptable salt thereof in the first layer.

In another aspect, the first layer may comprise promethazine hydrochloride and the multi-layered tablet may provide a plasma concentration within a therapeutic range of the at least one drug in the second layer over a period which is coextensive with at least about 80% of the period over which the multi-layered tablet provides a plasma concentration within a therapeutic range of promethazine hydrochloride.

In a still further aspect of the multi-layered tablet, the at least one drug in the second layer may comprise one or more of phenylepherine, pseudoephedrine, chlorpheniramine and pharmaceutically acceptable salts thereof.

The present invention also provides a liquid dosage form which comprises (a) promethazine and/or a pharmaceutically acceptable salt thereof and (b) at least one drug which is selected from decongestants, expectorants, mucus thinning drugs, antitussives, analgesics and antihistamines. This liquid dosage form provides a plasma concentration within the therapeutic range of component (b) over a period which is coextensive with at least about 70% of the period over which the liquid dosage form provides a plasma concentration within the therapeutic range of component (a).

In one aspect, the liquid dosage form may comprise a suspension.

In another aspect, at least a part of component (a) and/or of component (b) may be present as a complex with a complexing agent. For example, the complexing agent may comprise an ion-exchange resin such as, e.g., (sodium) polystyrene sulfonate.

In a still further aspect, the suspension may comprise particles of a complex of at least a part of component (b) with an ion-exchange resin, which particles are provided, at least in part, with a controlled release coating. This controlled release coating may comprise an organic polymer, e.g., methacrylate polymers.

The present invention also provides a method of concurrently alleviating (e.g., treating) a condition which can be alleviated by administration of promethazine and at least one other condition which can be alleviated by administration of a drug which is a decongestant, antitussive, expectorant, mucus thinning drug, analgesic and/or antihistamine. The method comprises administering any of the pharmaceutical dosage forms discussed above, including the various aspects thereof, to a subject in need thereof.

In one aspect of the method, the condition which can be alleviated by administration of promethazine comprises an allergic reaction.

In another aspect, the dosage form is preferably administered not more than about 3 times per day, e.g., twice per day.

The present invention also provides a process for making any of the pharmaceutical dosage forms discussed above, including the various aspects thereof. This method comprises the preparation of a first composition which comprises promethazine and/or a pharmaceutically acceptable salt thereof and the preparation of a second composition which comprises at least one second drug, and the combining of the first and the second compositions to form the dosage form.

In one aspect of the process, the first and second compositions may be combined by using a tablet press.

The present invention furthermore provides a pharmaceutical dosage form which comprises (a) a first drug which is an antihistamine and has a first plasma half-life and (b) at least one second drug which is selected from decongestants, antitussives, expectorants, mucus thinning drugs, analgesics and antihistamines and has a second plasma half-life which differs from the first plasma half-life by at least about 3 hours. This dosage form provides a plasma concentration within a therapeutic range of the at least one second drug over a period which is coextensive with at least about 70% of the period over which the dosage form provides a plasma concentration within the therapeutic range of the first drug.

In one aspect, the first half-life may be longer by at least about 4 hours than the half-life of the at least one second drug.

In another aspect, the period of a plasma concentration within the therapeutic range of the at least one second drug may be coextensive with at least about 80% of the period over which the dosage form provides a plasma concentration within the therapeutic range of the first drug.

In yet another aspect, the dosage form may comprise a bi-layered tablet.

In a still further aspect of the dosage form, the first plasma half-life may be at least about 8 hours.

In another aspect, the dosage form may be associated with instructions to administer the dosage form about three of fewer times per day, e.g., 1, 2 or 3 times per day.

The pharmaceutical dosage form which constitutes one aspect of the present invention comprises a first drug which is selected from promethazine and pharmaceutically acceptable salts thereof. The preferred salt of promethazine is the hydrochloride. However, other pharmaceutically acceptable salts of promethazine may be used as well. The term “pharmaceutically acceptable salt” as used herein and in the appended claims refers to those salts of a particular drug that are not substantially toxic at the dosage administered to achieve the desired effect and do not independently possess significant pharmacological activity. The salts included within the scope of this term are pharmaceutically acceptable acid addition salts of a suitable inorganic or organic acid. Non-limiting examples of suitable inorganic acids are, for example hydrochloric, hydrobromic, sulfuric and phosphoric acids. Non-limiting examples of suitable organic acids include carboxylic acids, such as acetic, propionic, tannic, glycolic, lactic, pyruvic, malonic, succinic, fumaric, malic, tartaric, citric, cyclamic, ascorbic, maleic, hydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acids, as well as sulfonic acids, such as methanesulfonic, ethanesulfonic, and β-hydroxyethanesulfonic acids.

In addition to the promethazine and/or pharmaceutically acceptable salt thereof, the dosage form contains one or more (e.g., one, two or three) second drugs. Preferred, non-limiting examples of such second drugs are decongestants (such as, e.g., phenylepherine, pseudoephedrine and pharmaceutically acceptable salts thereof), antitussives (such as, e.g., codeine, dihydrocodeine, hydrocodone, dextromethorphan and pharmaceutically acceptable salts thereof), expectorants and mucus thinning drugs (such as, e.g., guaifenesin), analgesics (such as, e.g., aspirin, acetaminophen, ibuprofen, ketoprofen, naproxen, sodium naproxen, meloxicam, hydrocodone, oxycodone, morphine, meperidine, and fentanyl) and antihistamines (such as, e.g., chlorpheniramine, carbinoxamine and pharmaceutically acceptable salts thereof).

The dosage form provides a plasma concentration within the therapeutic range of the at least one second drug over a period which is coextensive with (overlaps) at least about 70%, more preferred at least about 80%, e.g., at least about 90%, at least about 95%, or about 100%, of the period over which the dosage form provides a plasma concentration within the therapeutic range of the promethazine and/or salt thereof. The term “therapeutic range” as used herein and in the appended claims refers to the range of drug levels (including active metabolite levels) within which most patients will experience a significant therapeutic effect (including alleviation of symptoms) without an undesirable degree of adverse reactions. It is noted that the term “coextensive with” does not exclude, but rather includes, cases where a part of the period over which the plasma concentration of the at least one second drug (and/or active metabolites thereof) is within the therapeutic range is outside the period over which the plasma concentration of the promethazine and/or salt thereof is within the therapeutic range. In other words, even if the corresponding period for the at least one second drug is to overlap, for example, 70% of the corresponding period of the first drug, a certain percentage (preferably not more than about 30%, e.g., not more than about 20%, not more than about 10% or even not more than about 5%) of the total period over which the plasma concentration of the at least one second drug is within the therapeutic range may be outside the period over which the plasma concentration of the promethazine and/or salt thereof is within the therapeutic range.

The period over which the therapeutic range of a particular drug may be provided in a given case depends, at least in part, on the plasma half-life of the drug and/or active metabolites thereof. The term “plasma half-life” as used herein and in the appended claims refers to the time required for the plasma drug concentration to decline by 50%. The shorter the plasma half-life of a particular drug, the shorter will be the period within the therapeutic range of the drug which is provided by a single administered dose of the drug. In one preferred aspect of the dosage form of the present invention, the plasma half-life of the at least one second drug will be shorter than the plasma half-life of the promethazine and/or salt thereof by at least about 3 hours, e.g., by at least about 4 hours, by at least about 5 hours, by at least about 6 hours, by at least about 8 hours, or even by at least about 10 hours.

A preferred, although non-limiting, embodiment of the dosage form of the present invention is a tablet, in particular, a bi-layered tablet. Non-limiting examples of other embodiments of the dosage form of the invention are capsules, pills, chewable tablets, suspensions, solutions, syrups, and suppositories.

The bi-layered tablet which forms another aspect of the present invention comprises two layers. The first layer comprises promethazine and/or a pharmaceutically acceptable salt thereof, as discussed above. The second layer comprises at least one additional drug which is selected from decongestants, antitussives, expectorants, mucus thinning drugs, analgesics and antihistamines. Specific and non-limiting examples of such drugs are given above. The bi-layered tablet provides a plasma concentration within the therapeutic range of the at least one additional drug over a period which is coextensive with at least about 70%, preferably at least about 80%, e.g., at least about 90% or even about 100% of the period over which the bi-layered tablet provides a plasma concentration within the therapeutic range of the promethazine and/or pharmaceutically acceptable salt thereof.

In a preferred aspect of the bi-layered tablet, the promethazine and/or pharmaceutically acceptable salt thereof is the only active ingredient in the first layer. The second layer will usually contain one, two, three or even more additional drugs.

In another preferred aspect of the bi-layered tablet, the first layer is an immediate release layer and the second layer is a controlled release layer. The term “controlled release layer” as used herein and in the appended claims refers to any layer that is not an immediate release layer, i.e., does not release all of the active ingredients contained therein within a relatively short time (for example, within less than 1 hour, e.g., less than 0.5 hours, following ingestion of the dosage form). Accordingly, this term is a generic term which encompasses, e.g., sustained (extended) release layers, pulsed release layers, delayed release layers, and the like. Preferably, the controlled release layer releases the one or more active ingredients contained therein continuously or intermittently and, preferably, in approximately equal amounts per time unit, over an extended period of time such as, e.g., at least about 2 hours, at least about 3 hours, at least about 4 hours, or at least about 6 hours. The desirable length of the time period of continuous or intermittent (e.g., pulsed) release depends, inter alia, on the plasma half-life of the drug and/or an active metabolite thereof.

The first layer of the bi-layered tablet of the present invention will usually contain at least about 5 mg, e.g., at least about 8 mg, at least about 12 mg, or at least about 25 mg of promethazine hydrochloride or an equivalent amount of promethazine and/or any other pharmaceutically acceptable salt thereof. Usually, the first layer will not contain more than about 90 mg, e.g., not more than about 70 mg, not more than about 60 mg, or not more than about 50 mg of promethazine hydrochloride or an equivalent amount of promethazine and/or any other pharmaceutically acceptable salt thereof.

The second layer of the bi-layered tablet preferably is a controlled release layer, in particular, a sustained release layer. The controlled release layer may contain, by way of non-limiting example, (i) chlorpheniramine maleate, usually in an amount which is not less than about 0.1 mg, e.g., not less than about 2 mg, or not less than about 4 mg, but not more than about 16 mg, e.g., not more than about 12 mg, or equivalent amounts of any other pharmaceutically acceptable salt of chlorpheniramine; and/or (ii) phenylepherine hydrochloride, usually in an amount which is not less than about 1 mg, e.g., not less than about 10 mg, or not less than about 15 mg, but not more than about 90 mg, e.g., not more than about 50 mg, or not more than about 25 mg, or equivalent amounts of any other pharmaceutically acceptable salt of phenylepherine; and/or (iii) pseudoephedrine hydrochloride, usually in an amount which is not less than about 1 mg, e.g., not less than about 10 mg, not less than about 25 mg, or not less than about 50 mg, but not more than about 240 mg, e.g., not more than about 150 mg, not more than about 100 mg, or not more than about 70 mg, or equivalent amounts of any other pharmaceutically acceptable salt of pseudoephedrine.

Another aspect of the present invention is a multi-layered tablet which comprises at least a first layer and a second layer, but may optionally comprise a third, fourth, fifth, etc. layer. The first layer, which preferably is an immediate release layer, comprises promethazine and/or a pharmaceutically acceptable salt thereof (preferably as the only active ingredient contained therein) and the mandatory second layer is a controlled release layer and may comprise at least one drug which is selected from decongestants, antitussives, expectorants, mucus thinning drugs, analgesics and antihistamines. If more than one additional drug is to be incorporated in the tablet, the second layer may contain all of the additional drugs. Alternatively, a separate (third) layer may be provided for the second additional drug, for example, in cases where it would be difficult to design a controlled release layer which provides a desired release rate for both the first and the second additional drug. Of course, a fourth, fifth, etc. layer may be provided for a third or fourth additional drug, and so on. Alternatively and by way of non-limiting example, the second and a third layer may contain the same drug or drugs, but in different (relative) concentrations and/or incorporated in a different controlled release formulation.

The multi-layered tablet of the present invention will usually be made up of two or more distinct layers or discrete zones of granulation compressed together with the individual layers lying on top of one another. Layered tablets have the appearance of a sandwich because the edges of each layer or zone are exposed. Such conventional layered tablets are generally prepared by compressing a granulation onto a previously compressed granulation. The operation may be repeated to produce multi-layered tablets of more than two layers. In a preferred embodiment of the multi-layered tablet of the present invention, the tablet consists of two layers.

It is to be noted that it is not necessary for the two or more individual layers of the multi-layered tablet of the present invention to lie on top of one another. By way of non-limiting example, a second layer (e.g., sustained release layer) may be partially or completely surrounded by a first layer (e.g., an immediate release layer). For example, the second layer may be coated with the first layer. In the case of three layers, for example, the third layer may be partially or completely coated with the second layer, which in turn may be partially or completely coated with the first layer. Of course, these are but a few examples of the many different ways in which the various layers of the multi-layered tablet of the present invention can be arranged relative to each other. Moreover, it is to be understood that the tablets of the present invention are not limited to such multi-layered tablets. By way of non-limiting example, the tablet may comprise an immediate release matrix which comprises promethazine and/or pharmaceutically acceptable salt thereof, which matrix has dispersed therein particles of one or more sustained release formulations which have any of the other desired drug(s) incorporated therein.

In another aspect of the multi-layered tablet, the at least one drug in the second layer (and/or in the additional layers) may have a plasma half-life which is shorter by at least about 3 hours, e.g., shorter by at least about 4 hours, or shorter by at least about 6 hours, than the plasma half-life of promethazine and/or pharmaceutically acceptable salt thereof.

In another aspect of the multi-layered tablet, the tablet may provide a plasma concentration within a therapeutic range of the at least one drug in the second layer (e.g., one or more of phenylepherine, pseudoephedrine, chlorpheniramine and pharmaceutically acceptable salts thereof) over a period which is coextensive with at least about 80%, e.g., at least about 90%, of the period over which the multi-layered tablet provides a plasma concentration within the therapeutic range of the drug in the first layer, preferably of promethazine hydrochloride.

Another aspect of the present invention is formed by a liquid dosage form, preferably a suspension, which comprises (a) promethazine and/or a pharmaceutically acceptable salt thereof and (b) at least one drug which is selected from decongestants, expectorants, mucus thinning drugs, antitussives, analgesics and antihistamines. This liquid dosage form provides a plasma concentration within the therapeutic range of component (b) over a period which is coextensive with at least about 70%, preferably at least 80%, e.g., at least 90%, of the period over which the liquid dosage form provides a plasma concentration within the therapeutic range of component (a). This may be accomplished in various ways. By way of non-limiting example, component (b) may be incorporated into a solid controlled release formulation. For example, particles of component (b) may be provided with a controlled release coating (e.g. a controlled release coating comprising an organic polymer such as, e.g., a polyacrylate). This formulation may then be comminuted, if necessary, in an appropriate manner (e.g., by milling) to form particles of a size which is small enough to be suitable for being suspended in a pharmaceutically acceptable liquid carrier. Component (a), on the other hand, may be used as such or incorporated in a solid immediate release formulation, comminuted and incorporated into the liquid carrier as well. A non-limiting example of a corresponding procedure is described in the Examples below.

Prior to incorporating components (a) and (b) into a pharmaceutically acceptable liquid carrier to form a liquid dosage form according to the present invention, at least a part of component (a) and/or at least a part of component (b) may be transformed into a complex with a complexing agent. Non-limiting examples of suitable complexing agents comprise ion-exchange resins such as, e.g., (sodium) polystyrene sulfonate.

The dosage forms of the present invention can be manufactured by processes which are well known to those of skill in the art. For example, for the manufacture of bi-layered tablets, the active ingredients may be dispersed uniformly into a mixture of excipients, for example, by high shear granulation, low shear granulation, fluid bed granulation, or by blending for direct compression. Excipients may include diluents, binders, disintegrants, dispersants, lubricants, glidants, stabilizers, surfactants and colorants. Diluents, also termed “fillers”, are typically used to increase the bulk of a tablet so that a practical size is provided for compression. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders impart cohesive qualities to a tablet formulation and are used to ensure that a tablet remains intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants facilitate tablet manufacture; non-limiting examples thereof include magnesium stearate, calcium stearate, stearic acid, glyceryl behenate, and polyethylene glycol. Disintegrants facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Non-limiting examples of suitable glidants include silicon dioxide, talc and the like. Stabilizers inhibit or retard drug decomposition reactions, including oxidative reactions. Surfactants may be anionic, cationic, amphoteric or nonionic. If desired, the tablets may also contain minor amounts of nontoxic auxiliary substances such as pH buffering agents, preservatives, e.g., antioxidants, wetting or emulsifying agents, solubilizing agents, coating agents, flavoring agents, and the like.

Extended/sustained release formulations may be made by choosing the right combination of excipients that slow the release of the active ingredients by coating or temporarily bonding or decreasing the solubility of the active ingredients. Examples of these excipients include cellulose ethers such as hydroxypropylmethylcellulose (e.g., Methocel K4M), polyvinylacetate-based excipients such as, e.g., Kollidon SR, and polymers and copolymers based on methacrylates and methacrylic acid such as, e.g., Eudragit NE 30D.

There are several commercially available tablet presses capable of making bi-layered tablets. For example, Manesty RotaPress Diamond, a 45 station D tooling press, is capable of making bi-layered tablets described in this application. Non-limiting examples of presses for the manufacture of bi-layered tablets include Fette America Model No. PT 3090; Maneklal Global Exports (Mumbai, India) Models JD and DH series; Niro Pharma Systems, Model R292F; and Korsch AG Models XL 800 and XL 400.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those skilled in the art how the several forms of the present invention may be embodied in practice.

### EXAMPLE 1

### Liquid Formula

A liquid dosage form in accordance with the present invention which comprises promethazine hydrochloride, dihydrocodeine bitartrate and phenylepherine hydrochloride is illustrated as follows:

| Ingredients | Per 5 mL | Per 425 L |
|---|---|---|
| Promethazine Hydrochloride USP | 12.5 mg | 1.063 kg |
| Dihydrocodeine Bitartrate USP | 10.0 mg | 0.850 kg |
| Phenylepherine Hydrochloride USP | 10.0 mg | 0.850 kg |
| Methyl Paraben USP | 9.0 mg | 0.765 kg |
| Propyl Paraben USP | 1.0 mg | 0.085 kg |
| Propylene Glycol USP | 259 mg | 22.016 kg |
| Saccharin Sodium USP | 3.18 mg | 0.270 kg |
| Citric Acid USP | 5.0 mg | 0.425 kg |
| Strawberry Flavor | 10 mg | 0.850 kg |
| Banana Flavor | 10 mg | 0.850 kg |
| Sorbitol Solution 70% USP | 3212.5 mg | 273.1 kg |
| Purified Water, as required to q.s. to | 5.0 mL | 425 L |

Manufacturing process for 425 L batch size: In a suitably sized stainless steel vessel, dissolve methyl paraben and propyl paraben in approximately 50 L of warm (about 45° C.), purified water. Add about half of the propylene glycol and mix for about 1 hr. In a separate 1000 L stainless steel tank equipped with a suitably sized agitator, add about 50 L of purified water. With the agitator on, add phenylepherine hydrochloride, promethazine hydrochloride, saccharin sodium and citric acid and dissolve. Add the previously prepared paraben/propylene glycol solution to the 1000 L tank. Rinse the first vessel with about 2 L of water and transfer the rinsate to the 1000 L tank. Add the remaining propylene glycol to a suitably sized stainless steel vessel and dissolve the strawberry and banana flavors. Transfer this to the 1000 L tank. Rinse the container with 2 L of purified water and transfer to the 1000 L tank. With the agitator on, add the sorbitol solution 70% to the 1000 L tank. In a suitably sized stainless steel vessel, dissolve the dihydrocodeine bitartrate in about 5 L of purified water and transfer to the 1000 L tank. Rinse the container with about 2 L of purified water and transfer to the 1000 L tank. Stop the agitator and let the solution stand for 15 minutes. QS to 425 L with purified water. Filter product through a 1 micron filter and fill in appropriately sized containers.

To make products with other antihistamines, decongestants, antitussives, or expectorants, one or more combinations of each of the ingredients in a range as described in Table 1 below can be made depending on the specific therapeutic effect desired.

### EXAMPLE 2

### Suspension Formula

A suspension formula in accordance with the present invention which comprises promethazine hydrochloride and phenylepherine tannate is illustrated as follows:

| Ingredients | g/100 mL | kg/batch |
|---|---|---|
| =120 g | =1000 kg | |
| Promethazine Hydrochloride | 0.500 | 4.167 |
| Phenylepherine Tannate | 0.800 | 6.667 |
| Silica, colloidal anhydrous, NF | 1.73 | 14.4 17 |
| Hydroxyethylcellulose, NF | 0.05 | 0.417 |
| Sorbitol Solution 70% (non-crystallizing), NF | 34.00 | 283.333 |
| Glycerol | 14.75 | 122.917 |
| Xylitol, NF | 16.00 | 133.333 |
| Sodium Citrate, USP | 2.00 | 16.667 |
| Saccharin Sodium cryst., USP, | 0.01 | 0.083 |
| Sodium Benzoate, NF | 0.15 | 1.250 |
| Citric Acid Monohydrate, USP | 0.16 | 1.333 |
| Strawberry Flavor | 0.15 | 1.250 |
| Banana Flavor | 0.15 | 1.250 |
| Purified Water | 49.55 | 412.917 |
| Total Amount | 120.000 g | 1000.000 kg |

Manufacturing process for 1000 kg batch: In a suitably sized stainless steel vessel, dissolve saccharin sodium, sodium benzoate, citric acid, and sodium citrate in approximately 50 L of warm (about 45 deg C.), purified water. In another large stainless steel drum mix the silica, promethazine hydrochloride and micronized phenylepherine tannate until a uniform and consistent mixture is obtained. In a separate 1000 L stainless steel tank equipped with a suitably sized homogenizer/disperser add about 100 L of purified water. With the homogenizer on, add the silica mixture containing phenylepherine tannate and promethazine hydrochloride. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes. Filter product through a 10 micron filter and fill in appropriately sized containers.

To make products with other antihistamines, decongestants, antitussives, or expectorants, one or more combinations of each of the ingredients in a range as described in Table 1 below can be made depending on the specific therapeutic effect desired.

### EXAMPLE 3

### Bi-Layered Tablet (Direct Compression)

A bi-layered tablet in accordance with the present invention which comprises promethazine hydrochloride in one layer and phenylepherine hydrochloride and chlorpheniramine maleate in the other layer is illustrated as follows:

| | | |
|---|---|---|
| | Weight/tablet | Weight/1 kg batch |
| Ingredients | (mg) | (in grams) |

Layer 1 (Immediate release)

| | | |
|---|---|---|
| Promethazine Hydrochloride | 25.0 | 45.5 |
| Silicified Microcrystalline | 114.0 | 207.3 |
| Cellulose | | |
| Sodium Starch Glycolate | 10.0 | 18.2 |
| Magnesium Stearate | 1.0 | 1.8 |

Layer 2 (Sustained release)

| | | |
|---|---|---|
| Phenylepherine HC1 | 20.0 | 36.4 |
| Chlorpheniramine Maleate | 8.0 | 14.5 |
| Lactose Monohydrate | 50.0 | 90.9 |
| Dicalcium Phosphate | 50.0 | 90.9 |
| Kollidon SR | 252.0 | 458.2 |
| Stearic acid | 15.0 | 27.3 |
| Magnesium Stearate | 5.0 | 9.1 |
| Total | 550.0 | 1000.0 |

Manufacturing Process
(a) Immediate release layer: Screen all ingredients through a USP sieve size # 30. Blend promethazine hydrochloride (45.5 gms), silicified microcrystalline cellulose (207.3 gms) and sodium starch glycolate (18.2 gms) in a twin shell blender for 20 minutes. Add magnesium stearate (1.8 gms), which acts as a lubricant, to the above blend and mix for 3 minutes.

(b) Sustained release layer: Screen all ingredients through a USP sieve size # 30. Preblend a portion of the Kollidon SR (145 gms) and all the chlorpheniramine maleate (14.5 gms) for 15 minutes. Add the remaining Kollidon SR (313.2 gms), phenylepherine hydrochloride (36.4 gms), lactose monohydrate (90.9 gms) and dicalcium phosphate (90.9 gms) to the above preblend and mix for an additional 20 minutes. Add stearic acid (27.3 gms) and magnesium stearate (9.1 gms) to the above blend and mix for three minutes.

Manufacture bi-layered tablets using a rotary bi-layer tablet press where in each tablet the immediate release layer is 150 mgs and the sustained release layer is 400 mgs.

By using the process described above, a bi-layered tablet of the following composition may be manufactured by using direct compression:

| | |
|---|---|
| | Weight/tablet |
| Ingredients | (mgs) |

Layer 1 (Immediate Release)

| | |
|---|---|
| Promethazine Hydrochloride | 50 |
| Silicified Microcrystalline Cellulose | 133.5 |
| Sodium Starch Glycholate | 15 |
| Magnesium Stearate | 1.5 |

Layer 2 (Sustained Release)

| | |
|---|---|
| Phenylepherine HCl | 20 |
| Chlorpheniramine Maleate | 8 |
| Lactose Monohydrate | 50 |
| Dicalcium Phosphate | 50 |
| Kollidon SR | 252 |
| Stearic Acid | 15 |
| Magnesium Stearate | 5 |
| Total | 600 |

### EXAMPLE 4

### Bi-Layered Tablet (Wet Granulation)

A bi-layered tablet in accordance with the present invention which comprises promethazine hydrochloride in one layer and pseudoephedrine hydrochloride and chlorpheniramine maleate in the other layer is illustrated as follows:

| | | |
|---|---|---|
| | Weight /tablet | Weight/1 kg |
| Ingredients | (mgs) | batch (gms) |

Layer 1 (Immediate release)

| | | |
|---|---|---|
| Promethazine Hydrochloride | 25.0 | 35.7 |
| Silicified Microcrystalline Cellulose | 111.0 | 158.6 |
| Povidone | 3.0 | 4.3 |
| Croscarmellose Sodium | 10.0 | 14.3 |
| Magnesium Stearate | 1.0 | 1.4 |

Layer 2 (Sustained release)

| | | |
|---|---|---|
| Pseudoephedrine HCl | 60.0 | 85.7 |
| Chlorpheniramine Maleate | 8.0 | 11.4 |
| Microcrystalline Cellulose (PH 102) | 30.0 | 42.9 |
| Lactose Monohydrate | 100.0 | 142.9 |
| Dicalcium Phosphate | 100.0 | 142.9 |
| Povidone | 15.0 | 21.4 |
| Methocel K4M Premium | 212.0 | 302.9 |
| Stearic Acid | 20.0 | 28.6 |
| Magnesium Stearate | 5.0 | 7.1 |
| Total | 700.0 | 1000.0 |

Manufacturing Process

(a) Immediate release layer: Screen all ingredients through a USP sieve size # 30. Blend promethazine hydrochloride (35.7 gms), silicified microcrystalline cellulose (158.6 gms) and croscarmellose sodium (14.3 gms) in a high shear mixer/granulator for 10 minutes. Granulate the above blend using a 30% povidone solution (4.3 gms povidone in 14.3 gms purified water). Dry the granulation until the loss on drying (LOD) is less than 2.0%. Screen the dried granulation through a USP sieve size # 14. Add the screened granulation and the prescreened magnesium stearate (1.4 gms) to the above blend and mix for 3 minutes.

(b) Sustained release layer: Screen all ingredients through a USP sieve size # 30. Blend the pseudoephedrine hydrochloride (87.5 gms), chlorpheniramine maleate (11.4 gms), microcrystalline cellulose PH 102 (42.9 gms), lactose monohydrate (142.9 gms), dicalcium phosphate (142.9 gms), Methocel K4M Premium (302.9 gms) and stearic acid (28.6 gms) in a high shear mixer/granulator for 10 minutes. Granulate the above blend using a 30% povidone solution (21.4 gms povidone in 71.3 gms purified water). Dry the granulation till the LOD is less than 2.0%. Screen granules through a USP sieve size # 14. Add granules and the prescreened magnesium stearate (7.1 gms) to the above blend and mix for 3 minutes.

Manufacture bi-layered tablets using a rotary bi-layer tablet press where in each tablet the immediate release layer is 150 mgs and the sustained release layer is 550 mgs.

By using the process described above, a bi-layered tablet of the following composition may be manufactured by using wet granulation:

| | |
|---|---|
| | Weight/tablet |
| Ingredients | (mgs) |

Layer 1 (Immediate Release)

| | |
|---|---|
| Promethazine Hydrochloride | 50 |
| Silicified Microcrystalline cellulose | 129.5 |
| Povidone | 4 |
| Croscarmellose sodium | 15 |
| Magnesium Stearate | 1.5 |

Layer 2 (Sustained Release)

| | |
|---|---|
| Pseudoephedrine HCl | 60 |
| Chlorpheniramine Maleate | 8 |
| Microcrystalline Cellulose 102 | 30 |
| Lactose Monohydrate | 100 |
| Dicalcium Phosphate | 100 |
| Povidone | 15 |
| Hydroxypropylmethylcellulose | 212 |
| Stearic Acid | 20 |
| Magnesium Stearate | 5 |
| Total | 750 |

The above examples illustrate how to manufacture a bi-layered tablet containing promethazine hydrochloride in one layer and a combination of an antihistamine and/or a decongestant and/or an antitussive and/or an expectorant. For the layer that does not contain promethazine hydrochloride, combinations of one or more each of the non-limiting examples of possible ingredients in an exemplary range as described in the following Table 1 can be made depending on the specific therapeutic effect desired.

TABLE 1

| | | | |
|---|---|---|---|
| | | Preferred | OTC |
| | Amount per | Amount per | Daily |
| Active ingredient | Tablet | Tablet | Dosage |

| | | | |
|---|---|---|---|
| ANTIHISTAMINES | | | |
| Azelastine hydrochloride | 0.1-2.0 mg | 0.125 mg | |
| Azatadine hydrochloride | 0.1-4.0 mg | 1 mg | |
| Brompheniramine maleate | 0.1-64 mg | 2-16 mg | 24 mg |
| Dexbrompheniramine maleate | 0.1-24 mg | 3-6 mg | 12 mg |
| Carbinoxamine maleate | 0.1-16 mg | 4 mg | |
| Cetirizine hydrochloride | 0.1-40 mg | 5-10 mg | |
| Chlorcyclizine | 0.1-300 mg | | 75 mg |
| Chlorpheniramine maleate | 0.1-64 mg | 2-16 mg | 24 mg |
| Chlorpheniramine polistirex | 0.1-32 mg | 4-8 mg | |
| Clemastine | 0.1-12 mg | 0.5-2.68 mg | |
| Cyproheptadine | 0.1-16 mg | 2-4 mg | |
| Dexchlorpheniramine maleate | 0.1-24 mg | 2 mg | 12 mg |
| Cyproheptadine hydrochloride | 0.1-32 mg | 2-4 mg | |
| Diphenhydramine hydrochloride | 0.1-300 mg | 10-50 mg | 300 mg |
| Diphenhydramine citrate | 0.1-2000 mg | | 456 mg |
| Bromodiphenhydramine | 0.1-200 mg | 12.5-25 mg | |
| hydrochloride | | | |
| Doxylamine succinate | 0.1-200 mg | 12.5-25 mg | 75 mg |
| Fexofenadine hydrochloride | 0.1-720 mg | 30-180 mg | |
| Hydroxyzine hydrochloride | 0.1-400 mg | 10-100 mg | |
| Hydroxyzine pamoate | 0.1-400 mg | 25-100 mg | |
| Loratadine | 0.1-80 mg | 1-10 mg | |
| Desloratadine | 0.1-40 mg | 5 mg | |
| Phenindamine tartrate | 0.1-750 mg | | 150 mg |
| Pheniramine maleate | 0.1-750 mg | | 150 mg |
| Pyrilamine maleate | 0.1-200 mg | 25 mg | 200 mg |
| Terfenadine | | | |
| Thenyldiamine | | | |
| Thonzylamine | 0.1-3000 mg | | 600 mg |
| Thymol | | | |
| Tripelennamine hydrochloride | 0.1-400 mg | 25-50 mg | |
| Triprolidine hydrochloride | 0.1-40 mg | 1.25-5 mg | 10 mg |
| ANTITUSSIVES | | | |
| Chlorphedianol hydrochloride | 0.1-800 mg | | 100 mg |
| Codeine | 0.1-240 mg | 8.4-60 mg | 120 mg |
| Codeine phosphate | 0.1-240 mg | 2.5-60 mg | 120 mg |
| Codeine sulfate | 0.1-480 mg | | 120 mg |
| Dextromethorphan | 0.1-480 mg | | 120 mg |
| Dextromethorphan | 0.1-240 mg | 3.3-30 mg | 120 mg |
| hydrobromide | | | |
| Dextromethorphan polistirex | 0.1-240 mg | 30 mg | |
| Diphenhydramine citrate | 0.1-1000 mg | | 228 mg |
| Diphenhydramine hydrochloride | 0.1-400 mg | 10-50 mg | 150 mg |
| Benzonatate | 0.1-800 mg | 100-200 mg | |
| Hydrocodone bitatrate | 0.1-40 mg | 1.66-10 mg | |
| Dihydrocodeine | 0.1-128 mg | 16-32 mg | |
| Caramiphen edisylate | 0.1-160 mg | 6.7-40 mg | |
| Carbetapentane tannate | 0.1-480 mg | 30-60 mg | |
| Carbetapentane citrate | 0.1-160 mg | 20 mg | |
| Hydromorphone | 0.1-8 mg | 1 mg | |
| Noscapine | 0.1-200 mg | | |
| EXPECTORANT | | | |
| Guaifenesin | 0.1-2000 mg | 50-1200 | 2400 mg |

### REFERENCE EXAMPLE

### Extended Release Suspension

| Ingredients | Amount / 5 ml |
|---|---|
| Hydrocodone ion-exchange complex | Equivalent to 8 mgs Hydrocodone |
| | bitartarate |
| Dexchlorpheniramine ion-exchange | Equivalent to 4 mgs |
| complex | Dexchlorpheniramine maleate |
| Phenylephrine ion-exchange complex | Equivalent to 10 mgs |
| | Phenylephrine HCl |
| Eudragit ®L 100 | 0.2 to 2.8 grams |
| Glycerin | 315 mgs |
| Polysorbate 80 | 1.5 mgs |
| Carbomer (e.g.,Carbopol ®974) | 15 mgs |
| Methyl Paraben | 9 mgs |
| Propyl Paraben | 1 mgs |
| Artificial grape flavor | 5 mgs |
| FD&C red # 40 dye | 0.5 mgs |
| Water | q.s |

The formula described above serves as a non-limiting example. Any active drug which is in the form of a salt, such as promethazine hydrochloride, codeine phosphate, pseudoephedrine hydrochloride, morphine sulfate, or meperidine hydrochloride can be incorporated as an ion-exchange resin complex.

Procedure:

(1) Add the appropriate amount of sodium polystyrene sulphonate USP (e.g. Amberlite® IRP 69) to a hydrocodone bitartarate, dexchlorpheniramine maleate and phenylepherine HCl solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eugragit® L 100, Kollidon® MAE, Aquacoa®t cPD) and dry the granules.

(5) Mill the granules, if needed.

(6) To an appropriate amount of water add the following ingredients and dissolve: Carbomer (e.g., Carbopol® 974), glycerin, polysorbate 80, methyl paraben, propyl paraben, artificial grape flavor, FD&C red # 40 dye.

(7) Add milled granules.

(8) Add water to make up to a final volume.

(9) Agitate at suitable rate to avoid settling of the suspension and maintain a homogeneous product mixture.

(10) Fill in suitable containers ensuring that the product is homogeneous throughout the filling operation.

It is noted that the foregoing examples have been provided merely for the purpose of explanation and are in no way to be construed as limiting of the present invention. While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

1. A pharmaceutical dosage form which comprises (a) a first drug which is at least one of promethazine and a pharmaceutically acceptable salt thereof and (b) at least one second drug, wherein the dosage form provides a plasma concentration within a therapeutic range of the at least one second drug over a period which is coextensive with at least about 70% of a period over which the dosage form provides a plasma concentration within a therapeutic range of the first drug.

2. The dosage form of claim 1, wherein the at least one second drug is selected from decongestants, antitussives, expectorants, mucus thinning drugs, analgesics and antihistamines.

3. The dosage form of claim 2, wherein the first drug comprises promethazine hydrochloride.

4. The dosage form of claim 1, wherein the at least one second drug comprises an antitussive.

5. The dosage form of claim 4, wherein the antitussive comprises at least one of codeine, dihydrocodeine, hydrocodone, dextromethorphan and pharmaceutically acceptable salts thereof.

6. The dosage form of claim 1, wherein the at least one second drug comprises a decongestant.

7. The dosage form of claim 6, wherein the second drug comprises at least one of phenylepherine, pseudoephedrine and pharmaceutically acceptable salts thereof.

8. The dosage form of claim 1, wherein the at least one second drug comprises an antihistamine.

9. The dosage form of claim 8, wherein the antihistamine comprises at least one of chlorpheniramine and pharmaceutically acceptable salts thereof.

10. The dosage form of claim 1, wherein the at least one second drug comprises an expectorant.

11. The dosage form of claim 10, wherein the expectorant comprises guaifenesin.

12. The dosage form of claim 2, wherein a plasma half-life of the at least one second drug is shorter than a plasma half-life of the first drug by at least about 3 hours.

13. The dosage form of claim 1, wherein a plasma half-life of the at least one second drug is shorter than the plasma half-life of the first drug by at least about 4 hours.

14. The dosage form of claim 12, wherein the plasma half-life of the at least one second drug is shorter than the plasma half-life of the first drug by at least about 6 hours.

15. The dosage form of claim 1, wherein the period of a plasma concentration within the therapeutic range of the at least one second drug is coextensive with at least about 80% of the period of a plasma concentration within the therapeutic range of the first drug.

16. The dosage form of claim 12, wherein the period of a plasma concentration within the therapeutic range of the at least one second drug is coextensive with at least about 90% of the period of a plasma concentration within the therapeutic range of the first drug.

17. The dosage form of claim 2, wherein the period of a plasma concentration within the therapeutic range of the at least one second drug is coextensive with at least about 95% of the period of a plasma concentration within the therapeutic range of the first drug.

18. The dosage form of claim 1, wherein the dosage form comprises a tablet.

19. The dosage form of claim 18, wherein the tablet has at least two layers.

20. The dosage form of claim 19, wherein the tablet is a bi-layered tablet.

21. The dosage form of claim 18, wherein the tablet comprises a matrix which comprises the first drug and has dispersed therein particles which comprise the at least one second drug.

22. The dosage form of claim 21, wherein the matrix has dispersed therein particles which comprise a second drug and a third drug.

23. The dosage form of claim 21, wherein the matrix has dispersed therein particles which comprise a second drug, a third drug and a fourth drug.

24. The dosage form of claim 21, wherein the matrix provides an immediate release of the first drug and the particles provide a controlled release of the at least one second drug.

25. The dosage form of claim 1, wherein the dosage form comprises one of a solution and a suspension.

26. The dosage form of claim 25, wherein the dosage form comprises a suspension having particles therein which provide a controlled release of the at least one second drug.

27. A bi-layered tablet which comprises a first layer and a second layer, the first layer comprising a first drug which is at least one of promethazine and a pharmaceutically acceptable salt thereof, and the second layer comprising at least one second drug which is selected from decongestants, antitussives, expectorants, mucus thinning drugs, analgesics and antihistamines, wherein the bi-layered tablet provides a plasma concentration within a therapeutic range of the at least one second drug over a period which is coextensive with at least about 70% of a period over which the bi-layered tablet provides a plasma concentration within a therapeutic range of the first drug.

28. The bi-layered tablet of claim 27, wherein the second layer comprises at least one of phenylepherine, pseudoephedrine, chlorpeniramine and pharmaceutically acceptable salts thereof.

29. The bi-layered tablet of claim 28, wherein the first layer comprises promethazine hydrochloride and the second layer comprises at least two of phenylepherine, pseudoephedrine, chlorpeniramine and pharmaceutically acceptable salts thereof.

30. The bi-layered tablet of claim 27, wherein the first layer comprises only promethazine or a pharmaceutically acceptable salt thereof as an active ingredient.

31. The bi-layered tablet of claim 29, wherein the first layer comprises only promethazine hydrochloride as an active ingredient.

32. The bi-layered tablet of claim 30, wherein the period of a plasma concentration within the therapeutic range of the at least one second drug is coextensive with at least about 80% of the period of a plasma concentration within the therapeutic range of the first drug.

33. The bi-layered tablet of claim 31, wherein the period of a plasma concentration within a therapeutic range of the at least one second drug is coextensive with at least about 90% of the period of a plasma concentration within a therapeutic range of the first drug.

34. The bi-layered tablet of claim 30, wherein the first layer is an immediate release layer.

35. The bi-layered tablet of claim 27, wherein the second layer is a controlled release layer.

36. The bi-layered tablet of claim 34, wherein the first layer contains from about 0.1 mg to about 90 mg of promethazine hydrochloride.

37. The bi-layered tablet of claim 36, wherein the first layer contains from about 25 mg to about 50 mg of promethazine hydrochloride.

38. The bi-layered tablet of claim 36, wherein the second layer is a controlled release layer and contains at least one of (i) from about 0.1 mg to about 16 mg of chlorpheniramine maleate or an equivalent amount of at least one other pharmaceutically acceptable salt of chlorpheniramine; (ii) from about 1 mg to about 90 mg of phenylepherine hydrochloride or an equivalent amount of at least one other pharmaceutically acceptable salt of phenylepherine; and (iii) from about 1 mg to about 240 mg of pseudoephedrine hydrochloride or an equivalent amount of at least one other pharmaceutically acceptable salt of pseudoephedrine.

39. A multi-layered tablet which comprises at least a first layer and a second layer, wherein the first layer comprises at least one of promethazine and a pharmaceutically acceptable salt thereof and the second layer is a controlled release layer and comprises at least one drug which is selected from decongestants, antitussives, expectorants, mucus thinning drugs, analgesics and antihistamines.

40. The multi-layered tablet of claim 39, wherein the first layer is an immediate release layer.

41. The multi-layered tablet of claim 40, wherein the first layer comprises promethazine hydrochloride.

42. The multi-layered tablet of claim 39, wherein the first layer does not contain any active ingredient which is different from promethazine or a pharmaceutically acceptable salt thereof.

43. The multi-layered tablet of claim 39, wherein the second layer comprises at least one of codeine, dihydrocodeine, hydrocodone, dextromethorphan, phenylepherine, pseudoephedrine, guaifenesin, chlorpheniramine and pharmaceutically acceptable salts thereof.

44. The multi-layered tablet of claim 40, wherein the second layer comprises at least two of codeine, dihydrocodeine, hydrocodone, dextromethorphan, phenylepherine, pseudoephedrine, guaifenesin, chlorpheniramine and pharmaceutically acceptable salts thereof.

45. The multi-layered tablet of claim 39, wherein the at least one drug in the second layer has a plasma half-life which is shorter by at least about 3 hours than a plasma half-life of the at least one of promethazine and a pharmaceutically acceptable salt thereof in the first layer.

46. The multi-layered tablet of claim 39, wherein the first layer comprises promethazine hydrochloride and the tablet provides a plasma concentration within a therapeutic range of the at least one drug in the second layer over a period which is coextensive with at least about 80% of the period over which the multi-layered tablet provides a plasma concentration within a therapeutic range of promethazine hydrochloride.

47. The multi-layered tablet of claim 46, wherein the at least one drug in the second layer comprises one or more of phenylepherine, pseudoephedrine, chlorpheniramine and pharmaceutically acceptable salts thereof.

48. The multi-layered tablet of claim 39, wherein the layers are discrete zones which are arranged adjacent to each other.

49. The multi-layered tablet of claim 39, wherein the second layer is partially or completely surrounded by the first layer.

50. The multi-layered tablet of claim 49, wherein the second layer is coated with the first layer.

51. A liquid dosage form which comprises (a) at least one of promethazine and a pharmaceutically acceptable salt thereof and (b) at least one drug which is selected from decongestants, expectorants, mucus thinning drugs, antitussives, analgesics and antihistamines, wherein the liquid dosage form provides a plasma concentration within a therapeutic range of component (b) over a period which is coextensive with at least about 70% of a period over which the liquid dosage form provides a plasma concentration within a therapeutic range of component (a).

52. The liquid dosage form of claim 51, wherein the liquid dosage form comprises a suspension.

53. The liquid dosage form of claim 52, wherein at least a part of component (b) is present as a complex with a complexing agent.

54. The liquid dosage form of claim 53, wherein at least a part of component (a) is present as a complex with a complexing agent.

55. The liquid dosage form of claim 53, wherein the complexing agent comprises an ion-exchange resin.

56. The suspension of claim 55, wherein the ion-exchange resin comprises sodium polystyrene sulfonate.

57. The suspension of claim 53, wherein the suspension comprises particles of a complex of at least a part of component (b) with an ion-exchange resin, which particles are provided, at least in part, with a controlled release coating.

58. The suspension of claim 57, wherein the controlled release coating comprises an organic polymer.

59. The suspension of claim 58, wherein the organic polymer comprises a polyacrylate.

60. A method of concurrently alleviating a condition which can be alleviated by administration of promethazine and at least one other condition which can be alleviated by administration of a drug which is at least one of a decongestant, antitussive, expectorant, mucus thinning drug, analgesic and antihistamine, wherein the method comprises administering the pharmaceutical dosage form of claim 1 to a subject in need thereof.

61. A method of concurrently alleviating a condition which can be alleviated by administration of promethazine and at least one other condition which can be alleviated by administration of a drug which is at least one of a decongestant, antitussive, expectorant, mucus thinning drug, analgesic and antihistamine, wherein the method comprises administering the multi-layered tablet of claim 39 to a subject in need thereof.

62. The method of claim 61, wherein the condition which can be alleviated by administration of promethazine comprises an allergic reaction.

63. The method of claim 62, wherein the multi-layered tablet is administered not more than about three times per day.

64. The method of claim 61, wherein the multi-layered tablet is administered not more than about twice per day.

65. A method of concurrently alleviating a condition which can be alleviated by administration of promethazine and at least one other condition which can be alleviated by administration of a drug which is at least one of a decongestant, antitussive, expectorant, mucus thinning drug, analgesic and antihistamine, wherein the method comprises administering the liquid dosage form of claim 51 to a subject in need thereof.

66. A process of making the pharmaceutical dosage form of claim 1, wherein the method comprises preparing a first composition which comprises the first drug and a second composition which comprises the at least one second drug, and combining the first and the second compositions to form the dosage form.

67. The process of claim 66, wherein the first and second compositions are combined by using a tablet press.

68. A pharmaceutical dosage form which comprises (a) a first drug which is an antihistamine and has a first plasma half-life and (b) at least one second drug which is selected from decongestants, antitussives, expectorants, mucus thinning drugs, analgesics and antihistamines and has a second plasma half-life which differs from the first plasma half-life by at least about 3 hours, wherein the dosage form provides a plasma concentration within a therapeutic range of the at least one second drug over a period which is coextensive with at least about 70% of a period over which the dosage form provides a plasma concentration within a therapeutic range of the first drug.

69. The dosage form of claim 68, wherein the first plasma half-life is longer by at least about 4 hours than the plasma half-life of the at least one second drug.

70. The dosage form of claim 69, wherein the period of a plasma concentration within the therapeutic range of the at least one second drug is coextensive with at least about 80% of the period over which the dosage form provides a plasma concentration within the therapeutic range of the first drug.

71. The dosage form of claim 68, wherein the dosage form comprises a bi-layered tablet.

72. The dosage form of claim 70, wherein the first plasma half-life is at least about 8 hours.

73. The dosage form of claim 68, wherein the dosage form is associated with instructions to administer the dosage form three or fewer times per day.

74. The dosage form of claim 71, wherein the dosage form is associated with instructions to administer the dosage form three or fewer times per day.
